(19) United States
(12) Patent Application Publication
(10) Pub. No.: US 2025/0356689 A1
(43) Pub. Date: Nov. 20, 2025

(54) DATA-DRIVEN SURROGATE CIRCUMVENTION DETECTION

(71) Applicant: 1A Smart Start LLC, Grapevine, TX (US)

(72) Inventors: Toby Taylor, Flower Mound, TX (US); Mark Bellehumeur, Frisco, TX (US)

(73) Assignee: 1A Smart Start LLC, Grapevine, TX (US)

(21) Appl. No.: 19/210,158

(22) Filed: May 16, 2025

Related U.S. Application Data
(60) Provisional application No. 63/648,958, filed on May 17, 2024.

(51) Int. Cl. G06V 40/16; G01N 33/497

(52) U.S. Cl. CPC G06V 40/167; G01N 33/4972; G06V 40/172

(57) ABSTRACT

The technology described herein relates to methods and systems of detecting surrogate circumvention events based on a data-driven process. Common methods of circumvention include having a sober individual provide the breath sample or using various mechanical or electronic devices to mimic human breath (“surrogate samples”). In other instances, the assigned offender may drive a different vehicle (“surrogate vehicle”) other than the vehicle with the installed interlock device. The technology described herein records data events associated with breath tests to determine whether a surrogate circumvention event has likely occurred. The data events may include time-series sensor measurements from the BAIID and/or patterns of passed, failed, and skipped breath tests to predict the occurrence of the surrogate circumvention events.

### CROSS-REFERENCE TO RELATED APPLICATIONS

This application claims the benefit of U.S. Provisional Application No. 63/648,958 filed May 17, 2024, entitled “Data-Driven Surrogate Circumvention Detection,” which is incorporated herein by reference in its entirety.

### BACKGROUND

Breath Alcohol Ignition Interlock Devices (BAIIDs) are sophisticated tools installed in vehicles that require drivers to provide a breath sample before starting their engines or operating their vehicles (collectively “start” or “starting.”). The devices measure the driver's blood alcohol concentration (BAC) and prevents the vehicle from starting or operating if the driver's BAC exceeds a predetermined limit and are commonly used as a part of a DUI (Driving Under the Influence) program typically managed by a governmental entity. While BAIIDs have proven effective in reducing instances of impaired driving, there is a need to continuously improve their effectiveness.

It is with respect to these limitations and other considerations that examples have been made. In addition, although relatively specific problems have been discussed, it should be understood that the examples should not be limited to solving the specific problems identified in the background.

### SUMMARY

The technology described herein relates to methods and systems of detecting surrogate circumvention events based on a data-driven process. Common methods of circumvention include having a sober individual provide the breath sample or using various mechanical or electronic devices to mimic human breath (“surrogate samples”). In other instances, the assigned offender may drive a different vehicle (“surrogate vehicle”) other than the vehicle with the installed interlock device. The technology described herein records data events associated with breath tests to determine whether a surrogate circumvention event has likely occurred. The data events may include time-series sensor measurements from the BAIID and/or patterns of passed, failed, aborted, and skipped breath tests to predict the occurrence of the surrogate circumvention events.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

The present disclosure is illustrated by way of example by the accompanying figures, in which like references indicate similar elements. Elements in the figures are illustrated for simplicity and clarity and have not necessarily been drawn to scale.

FIG. 1 depicts an example system for data-driven surrogate circumvention detection.

FIG. 2 depicts additional components of the example system of FIG. 1.

FIGS. 3A-3B depict an example method of triggering a surrogate check based on interlock data.

FIG. 4 depicts another example method of triggering a surrogate check based on interlock data.

FIG. 5 depicts another example method of triggering a surrogate check based on interlock data.

FIG. 6 depicts an example method of triggering a surrogate vehicle alert.

FIG. 7 depicts a block diagram illustrating physical components of a computing device.

### DETAILED DESCRIPTION

As discussed briefly above, while Breath Alcohol Ignition Interlock Devices (BAIIDs) have proven effective in reducing instances of impaired driving, there is a need to continuously improve their effectiveness. Despite the effectiveness of BAIIDs, some individuals may attempt to circumvent the devices to start their vehicles despite being intoxicated. Common methods of circumvention include having a sober individual provide the breath sample or using various mechanical or electronic devices to mimic human breath (“surrogate samples”). These circumvention techniques pose a significant risk to public safety, as they allow potentially impaired drivers to operate vehicles and endanger lives.

The addition of cameras to BAIIDs provides an extra layer of security and validation. When a “triggering event” occurs (e.g., a breath sample or “violation”) the BAIID camera captures the driver's image simultaneously. This visual evidence confirms the individual providing the breath test is indeed the driver and not someone else attempting to start the vehicle.

The use of cameras with BAIIDs discourages attempts at manipulation and further reinforces the seriousness of drunk driving offenses. Moreover, the visual evidence recorded by the camera can serve as valuable information for monitoring authorities. In case of non-compliance or violations, such as the use of a surrogate, the image captured by the camera can be used in legal proceedings. This contributes to a more efficient legal process and helps ensure strict adherence to BAIID requirements.

Driver data from the BAIIDs and ancillaries provides a wealth of information regarding the methods used by individuals attempting to circumvent BAIIDs. By analyzing the data, patterns can be identified, revealing potential loopholes that can be addressed through technological advancements and system updates. This ongoing analysis assists in refining the design of BAIIDs and improving their effectiveness in preventing drunk driving incidents.

To effectively identify circumvention, algorithms require access to extensive datasets that encompass a wide range of breath samples collected under different conditions. These datasets can include information about normal breath patterns, average BAC levels, and specific characteristics associated with circumvention attempts. Machine learning algorithms can be trained using these datasets to recognize patterns and anomalies, improving their ability to detect potential circumvention.

Algorithms, with their ability to process vast amounts of data and detect patterns, can significantly enhance the effectiveness of BAIIDs by identifying attempts to circumvent. By analyzing breath samples and comparing them against pre-determined patterns, algorithms can detect inconsistencies or irregularities that may indicate circumvention. Furthermore, algorithms can also detect circumvention attempts by monitoring changes in device or vehicle behavior or unexpected breath patterns. The utilization of algorithms in conjunction with BAIIDs allows for proactive identification of circumvention, enabling appropriate action to be taken, and acts as a deterrent, as potential offenders will be aware of the advanced detection capabilities of the device, reducing the likelihood of circumvention attempts.

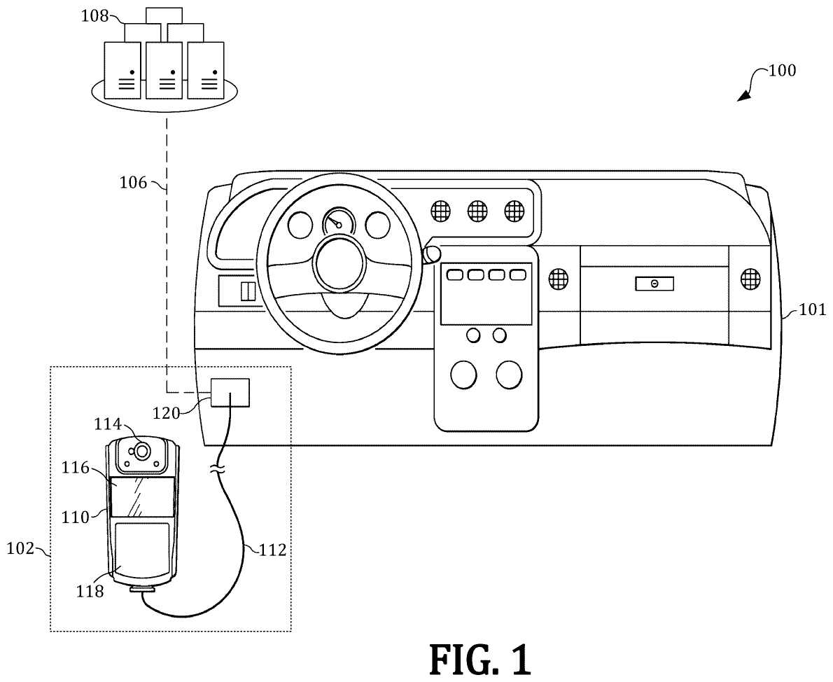

FIG. 1 depicts an example system 100 for data-driven circumvention detection. The system 100 includes BAIID 102 that includes a breath analyzer 110 and a controller 120 that is connected to a vehicle 101. The breath analyzer 110 in the current example is a handheld device that receives breath samples from a user of the BAIID 102, who is often the driver of the vehicle 101. In the example depicted, the breath analyzer 110 is connected to the controller 120 via a wired connection 112. In other examples, the breath analyzer may be connected to the controller 120 via a wireless connection. The controller 120 is connected to a vehicle 101, and the controller 120 controls the ability for the vehicle to start based the on alcohol content of a breath sample determined by the breath analyzer 110. For instance, if the user passes the breath test, the controller 120 allows the vehicle 101 to start. If the user fails the breath test, the controller 120 prevents the vehicle 101 from starting.

During operation, the BAIID 102 collects and generates data about the breath samples collected and/or operation of the vehicle 101. That data may be referred to herein as interlock data 106. The interlock data 106 is transmitted from the BAIID 102 to a remote server 108. The remote server 108 may then perform additional actions on the received interlock data 106, as discussed further herein. In some examples, the remote server 108 is operated by the same entity or company that operates the BAIID 102. In other examples, the remote server 108 is operated by a monitoring authority that monitors violations of offenders that have had the BAIID 102 installed in their respective vehicles 101. In examples where the remote server 108 is not operated by the monitoring authority, the remote server 108 may be in communication with the monitoring authority to send alerts and/or messages to the monitoring authority.

As some additional detail, the example breath analyzer 110 includes a mouthpiece 114, a display screen 116, and a user interface 118. The mouthpiece 114 may be removable from the breath analyzer 110. When in use, the breath analyzer 110 collects a breath sample via the mouthpiece 114. The display screen 116 displays instructions for use of the breath analyzer 110 as well as results determined by the breath analyzer 110. For instance, when a BAC value is determined, that value may be surfaced on the display screen 116. Whether the user has passed or failed a particular breath test may also be displayed on the display screen 116. The user may further interact with the breath analyzer 110 via the user interface 118, which may include a keypad or other suitable input interface.

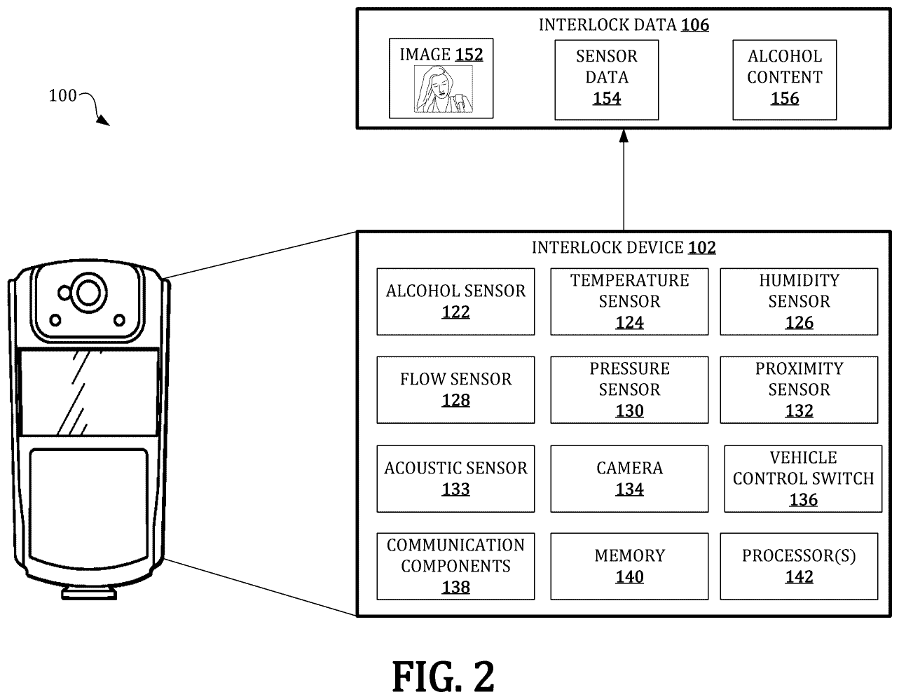

FIG. 2 depicts additional components of the example system 100 of FIG. 1. For instance, FIG. 2 depicts additional components of the BAIID 102. The example BAIID 102 includes an alcohol sensor 122, a temperature sensor 124, a humidity sensor 126, a flow sensor 128, a pressure sensor 130, a proximity sensor 132, an acoustic sensor 133, a camera 134, a vehicle control switch 136, communication components 138, memory 140, and at least one processor 142. In other examples, the BAIID 102 may include a greater or fewer number of components and/or sensors.

The alcohol sensor 122 detects an alcohol vapor concentration of the breath received by the example breath analyzer 110. The alcohol sensor 122 may be one of various forms of alcohol sensors, such as a fuel cell, the metal-oxide semiconductor, an infrared spectroscopy sensor, and/or a photoionization detector, among others. As one example, a fuel cell sensor may include a porous membrane that is coated with platinum, which oxidizes breath to produce an electrical current. The produced current is proportional to the alcohol concentration in the received breath. Thus, the produced current can be used to generate an alcohol content (e.g., BAC) for the user that delivered the breath.

The temperature sensor 124 measures the temperature of the received breath (e.g., the exhaled gas from the user). The measured temperature may be a time series of measurements over the received breath. For instance, the temperature sensor 124 may have a sampling rate of 30-50 milliseconds (ms) or less in some examples to allow for a time series of temperature measurements to be generated for the breath that is received. The other sensors discussed herein may have similar sampling rates.

The humidity sensor 126 measures the humidity of the received breath. The measured humidity may also be a time series of humidity measurements over the received breath.

The flow sensor 128 measures the flow of the breath that is received by the example BAIID 102. The measured flow may also be a time series of flow measurements over the received breath. The measured flow may be integrated over the time of the breath to determine the volume of the received breath.

The pressure sensor 130 measures the pressure of the received breath. The measured pressure may also be a time series of pressure measurements over the received breath.

The proximity sensor 132 measures the proximity of the user to the example breath analyzer 110. In some examples, the proximity sensor 132 is positioned to detect a position of the user's lips to the example breath analyzer 110, such as the proximity of the lips to the mouthpiece 114 or a portion thereof. In other examples, the proximity of a different portion of the user's face is detected by the proximity sensor 132. The measured proximity may also be a time series of proximity measurements over the received breath.

The acoustic sensor 133 measures acoustic frequencies or vibrations during the breath delivery. In some examples, the acoustic sensor 133 may be a microphone or similar device. The acoustic sensor 133 may capture different sounds made by the user of the breath analyzer 110 as the user delivers the breath for a breath test.

The camera 134 captures images of the user. The camera 134 may be integrated into the example breath analyzer 110 itself, and/or the camera 134 may be separate from the example breath analyzer 110 but in communication with the example breath analyzer 110. For instance, the camera 134 may be mounted on the dash or visor of the vehicle 101 and communicate the captured images to the example breath analyzer 110.

The vehicle control switch 136 provides control of the vehicle's starting capability. The vehicle control switch 136 may be in the form of a relay and/or be operated in software and/or firmware-based controls. The vehicle control switch 136 prevents the vehicle from starting when there is a failed breath test and/or a lack of a passed breath test.

The communication components 138 communicate with other computing devices. Examples of suitable communication components 138 include radio transmitters, receivers, transceiver circuitry, universal serial buses (USBs), parallel, and/or serial ports. Some specific examples include cellular communication equipment that allows for communication over cellular networks, such as 4G and/or 5G communication networks. Other examples include BLUETOOTH connection components and/or Wi-Fi connection components that allow for communications over the respective bands.

The memory 140 stores the sensor measurements and data captured by the example BAIID 102. The memory 140 also stores instructions that, when executed by the processor 142, cause the example BAIID 102 to perform the operations discussed herein. The memory 140 may include volatile storage (e.g., random access memory), non-volatile storage (e.g., read-only memory), flash memory, or any combination of such memories. For instance, the memory 140 may include random access memory (RAM), read-only memory (ROM), electrically erasable programmable ROM (EEPROM), flash memory, or other memory technology which can be used to store information.

The processor 142 may be a central processing unit (CPU) or other hardware logic that provides the processing capabilities discussed herein. The processor 142 may be a microprocessor, Field-Programmable Gate Arrays (FPGAs), Application-Specific Integrated Circuits (ASICs), Application-Specific Standard Products (ASSPs), System-on-a-Chip systems (SOCs), Complex Programmable Logic Devices (CPLDs), etc. The processor 142 executes instructions stored in memory 140 and generates control signals to the various components of the example BAIID 102.

The example BAIID 102 generates the interlock data 106, as discussed above. The interlock data 106 may include items such as a captured image 152 of the user captured by the camera 134, sensor data 154 captured by the sensors of the example BAIID 102, and/or the alcohol content 156 (e.g., BAC) determined by the example breath analyzer 110.

The interlock data 106 that is generated from the example BAIID 102 is then transmitted to the one or more remote servers 108. The interlock data 106 may be transmitted via the communication components 138 (e.g., via a cellular network). The interlock data 106 may be transmitted at the conclusion of each breath test. In other examples, the interlock data 106 may be transmitted at specified intervals and include data for multiple tests and/or captured breaths.

As should be appreciated, throughout the world there are many BAIIDs 102 installed in many vehicles 101. Hundreds of thousands of BAIIDs 102 are installed every year in the United States alone. Each of these BAIIDs 102 is then used for multiple breath tests per day-generating millions of discrete interlock data 106 being transmitted to the remote server(s) 108. Such massive amounts of data lead to large storage and processing requirements. For example, with each breath test, an image 152 is captured. Performing an image analysis, such as facial recognition, on each of the received images requires significant processing power and resources. However, many breath tests do not involve any nefarious activity or intent, such as an attempted surrogate circumvention. As a result, performing facial recognition on each of the millions of daily received images wastes resources for the breath tests where no circumvention is being attempted.

With the present technology, the patterns of the breath tests are analyzed to identify when a surrogate circumvention is likely being attempted. When there is an increased likelihood of a surrogate circumvention, a surrogate check may be performed by analyzing the images that were captured during the respective breath tests. When there is a low likelihood of a surrogate circumvention, such image analysis may be avoided, resulting in the conservation of additional computing resources.

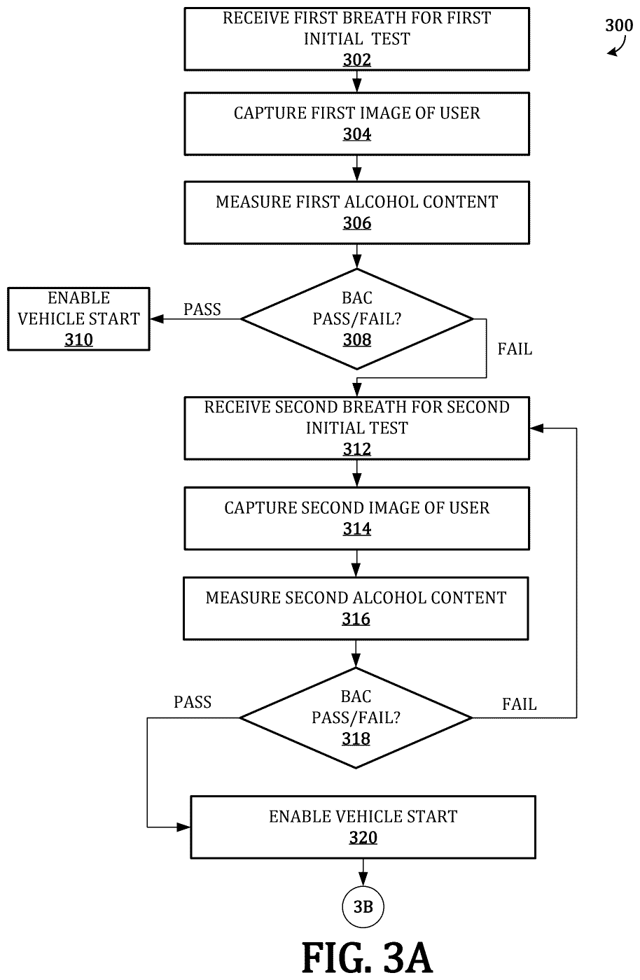

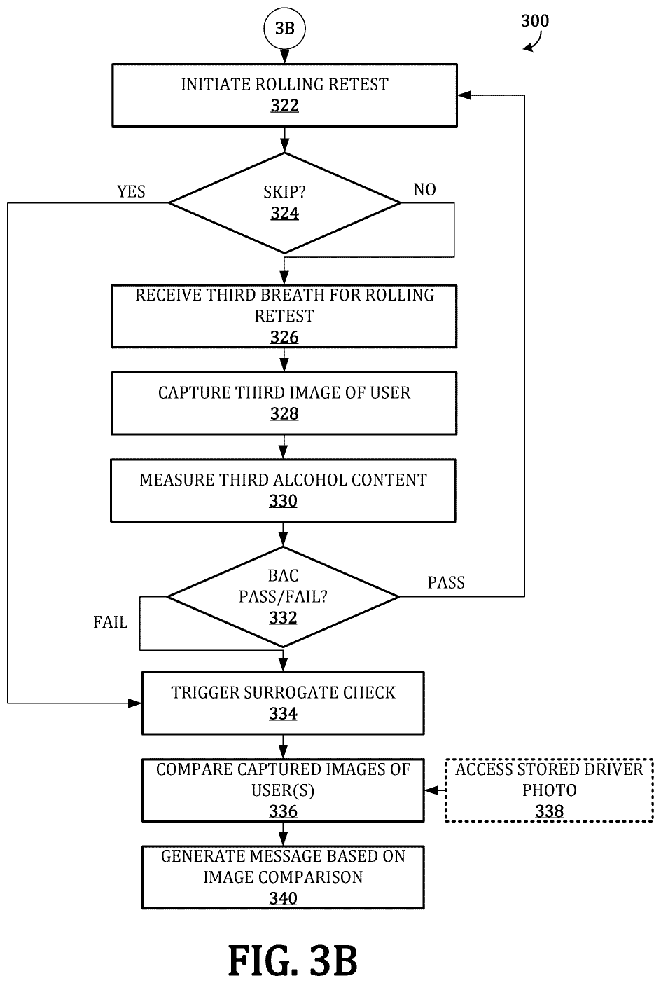

FIGS. 3A-3B depict an example method 300 of triggering a surrogate check based on interlock data. The method 300 analyzes a pattern of failed (or skipped) breath tests and passed breath tests to predict whether a surrogate circumvention occurred or was attempted. For example, the method predicts when a surrogate (e.g., non-driver) has delivered a passing breath sample to allow a different person to start or operate the vehicle equipped with the BAIID. The surrogate-circumvention prediction may be based on a particular sequence of breath tests, such as an initial failed breath test, followed by a passed breath test, then followed by a failed or skipped rolling retest. Upon detection of such a pattern, a surrogate check may be performed to determine if a surrogate delivered the breath for the passing breath test. Method 300 provides an example of such a process.

At operation 302, a first breath is received by a BAIID for a first initial test. The first initial test may be when a driver is first attempting to start the car. As part of the first initial test, a first image of the current user of the BAIID is captured at operation 304. At operation 306, the first alcohol content (e.g., BAC) of the received first breath is determined. The first alcohol content is determined using the alcohol sensor of the BAIID.

At operation 308, a determination is made as to whether the first alcohol content results in a pass or fail of the breath test. For instance, the first alcohol content is compared to a threshold amount, such as a BAC threshold of 0.08. If the first alcohol content is below the threshold, the first breath test is passed and the method 300 flows to operation 310. At operation 310, the BAIID enables the vehicle to start. If, however, the first alcohol content is at or above the threshold, the first breath test is failed and the method 300 flows to operation 312.

At operation 312, a second breath is received for a second initial test. For example, after the failed first initial test, a subsequent test may be attempted. This second initial test may be performed by the same user (e.g., the driver) or by another user (e.g., a surrogate). At operation 314, a second image is captured of the user delivering the second breath. At operation 316, a second alcohol content is measured for the second breath that was received in operation 312.

At operation 318, a determination is made as to whether the second alcohol content passes or fails the second initial breath test. Such a determination may be made by comparing the second alcohol content to the threshold (e.g., BAC of 0.08). If the second alcohol content exceeds the threshold, the second initial breath test fails. The method 300 may then flow back to operation 312 where yet another initial breath test may be attempted. In other examples, a violation may be reported and/or additional initial tests may be prevented for a set period of time.

If, at operation 318, the second alcohol content is below the threshold, the second initial breath test has passed and method 300 flows to operation 320. At operation 320, the BAIID enables the vehicle to start based on the passed second initial breath test.

The driver of the vehicle then proceeds to drive the vehicle for a period of time. At some point while the vehicle is being driven, the BAIID initiates a rolling retest at operation 322. A rolling retest is a safety feature that is designed to help ensure that the driver remains sober while operating the vehicle after passing the initial breath test to start the vehicle. The rolling retests may be initiated based on random time intervals and/or preset time intervals. Other criteria may also be used to initiate the rolling retest. Initiating the rolling retest may include generating an audible and/or visual notification via the BAIID indicating that a rolling retest is required. The notification may also indicate a set time frame in which the driver has to take the rolling retest (e.g., a few minutes). In some examples, the driver may ignore the alert and skip the test.

At operation 324, a determination is made as to whether the rolling retest has been skipped. The determination may be based on whether the rolling retest was completed within the set time period to complete the rolling retest. If the rolling retest is skipped, method 300 flows to operation 334 where a surrogate check is triggered. The surrogate check is discussed in more detail below. Skipping of the rolling retest may also result in an alert being recorded and/or transmitted indicating the skip of the rolling retest. If, at operation 324, the rolling retest is not skipped (e.g., the rolling retest begins within the time limit), method 300 flows to operation 326.

At operation 326, a third breath for the rolling retest is received by the BAIID. At operation 328, an image is captured of the user delivering the third breath as part of the rolling retest. At operation 330, a third alcohol content of the received third breath is measured by the BAIID for the rolling retest.

At operation 332, a determination is made as to whether the third alcohol content passes or fails the rolling retest. For example, the third alcohol content is compared to the alcohol threshold (e.g., BAC of 0.08). If the third alcohol content is lower than the threshold, the rolling retest is passed and the driver may continue driving (e.g., restart the vehicle). The method 300 then flows back to operation 322 where another rolling retest may be initiated at a random later time. If the third alcohol content is at or above the alcohol threshold, the rolling retest is failed, internal/external alarms signal the driver to turn the vehicle off, and the ability to restart the vehicle is disabled without providing a passing initial breath check that is lower than the threshold. The method 300 then flows to operation 334.

At operation 334, the surrogate check is triggered. The surrogate check may be triggered based on a particular pattern of failing the first initial breath check, passing the second initial breath check, and then failing or skipping the rolling retest. In some examples, time limits may be considered as further criteria for triggering the surrogate check. For instance, one temporal criterion may include that the failed first initial breath check and the passed second initial breath check must be within a first threshold timespan from one another (e.g., 5 minutes, 10 minutes, 15 minutes). Another temporal criterion may be that the passed second initial breath check and the failed or skipped rolling retest be within a second threshold timespan. The second threshold timespan is generally longer in duration than the first threshold timespan. For instance, the second threshold timespan may be at least double the first threshold timespan. In some examples, the second threshold timespan is between about 0-3 hours (e.g., 2 hours). Yet another temporal criterion may be that the failed first initial breath test, the passed second initial breath test, and the skipped or failed rolling retest all be within a third threshold timespan of one another (e.g., 1 hour, 2 hours, 3 hours).

Performance of the surrogate check may include performing operations 336-340 of method 300. For example, at operation 336, a comparison is performed with one or more of the images captured during method 300, such as the first image captured at operation 304, the second image captured at operation 314, and/or the third image captured at operation 328. For example, the second image may be compared to the first image to determine if the user in the second image is the same person as in the first image. The third image may also be compared to the first image and/or the second image to determine if the same user is in the respective images. If there are different users in the images, then a surrogate circumvention has been identified. This surrogate circumvention event may then be stored and/or reported to the monitoring authority.

The image comparison performed at operation 336 may also include comparing one or more of the captured images with a stored image of the driver to which the BAIID is assigned. In such examples, the stored photo of the driver is accessed at operation 338. Accessing the stored driver photo may include querying a database that stores the respective photo. The database may be located at the remote server and/or at another storage location remote from the remote server and/or the BAIID. Comparing the images may include comparing the captured first, second, and/or third image with the stored driver photo. In other examples, only the second image corresponding to the passed second initial test is compared to the stored driver photo. A determination is then made as to whether the face(s) in the captured images match the stored driver photo. If the face(s) do not match the stored driver photo and/or if the face in the photo for the passed test does not match the stored driver photo, a surrogate circumvention is identified. The surrogate circumvention event may then be stored and/or reported to the monitoring authority.

The comparison of the various images may be performed through the use of facial-recognition technology, such as by performing facial recognition algorithms on the images and comparing the resultant faces and/or facial features. Such facial recognition algorithms may first detect the presence of a face in the image. Detection of a face may be performed by using deep learning models, such as convolutional neural networks (CNNs), and/or Haar feature-based cascade classifiers, among other techniques. Features of the detected face are then extracted or identified, such as the corners of the eyes, tip of the nose, and/or corners of the mouth, among other possible features. Feature vectors for the extracted features of the detected face may then be generated. The feature vectors represent the unique characteristics of the face. The feature vectors for the face of one image may then be compared to the feature vectors for the face of another image to determine if the same face is present in the two images. This comparison may be performed via a cosine similarity analysis and/or Euclidean distance analysis (among other possible comparison functions) of the feature vectors to determine if the feature vectors are within a threshold distance. If the feature vectors are within the threshold distance, the faces are considered to be a match.

As an example in the context of method 300, a first face is identified in the first captured image. First feature vectors are generated for the first face. A second face is identified in the second captured image. Second feature vectors are generated for the second face. The first feature vectors are compared to the second feature vectors to determine if the first face and the second face belong to the same person. If the faces belong to different people, a surrogate circumvention is identified.

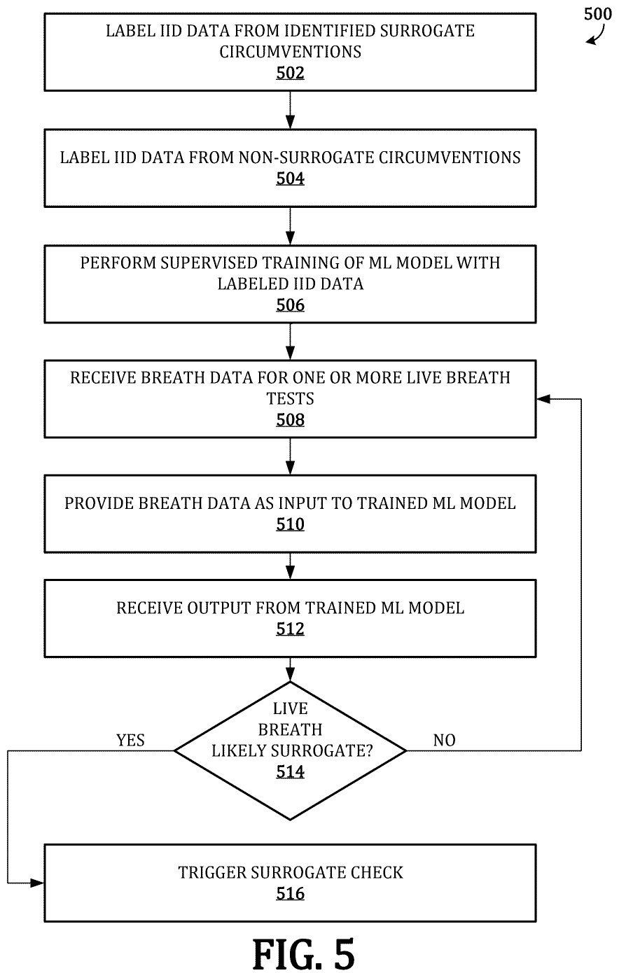

At operation 340, a message is generated based on the comparison of the images performed in operation 336. The message may be an alert to the monitoring authority when a surrogate circumvention has been identified. The message may include the times and results of the respective breath tests. The message may also include the captured images for the respective breath tests. The data captured for each of the breath tests of the circumvention events may also be stored for later use as training data for machine learning models, as discussed further below with respect to FIG. 5.

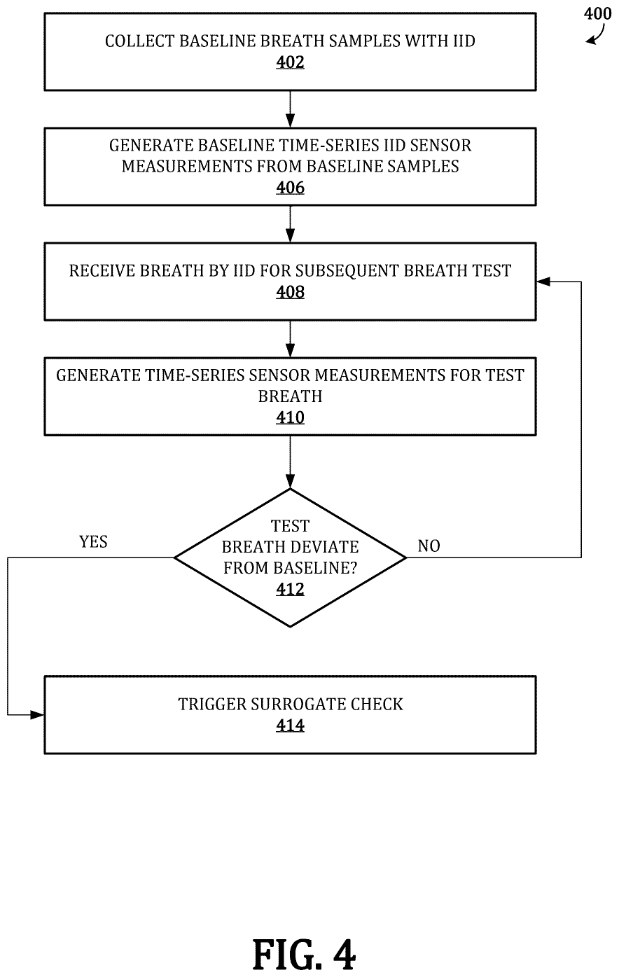

FIG. 4 depicts another example method 400 of triggering a surrogate check based on interlock data. Different people have different breathing patterns and parameters when performing a breath test. For instance, some people may breathe more quickly or forcefully at the beginning of the breath delivery and then taper the breath. Others may provide the opposite. The humidity and/or temperature levels throughout breath delivery may also vary uniquely from person to person. These unique trends and traits may be used to distinguish one person from another. While the traits are likely not as unique as a fingerprint, the traits are often unique enough to distinguish an assigned driver from a surrogate that is delivering a breath. Method 400 provides an example process of identifying a surrogate circumvention based on traits of the breath delivery during a breath test.

At operation 402, a set of baseline breath samples are collected with a BAIID. This baseline collection of breath samples may occur at the time that the BAIID is installed in the vehicle of the offender or within a short time (several days) thereafter. The collection may be performed in a supervised environment where a human can observe the person delivering the breath and verify that it is the offender assigned to the BAIID that is actually delivering the breaths. In other examples, images of the user may be captured during the collection and used to verify that the user delivering the baseline breath samples is the assigned offender.

At operation 406, baseline time-series sensor measurements are generated from the baseline breath samples collected in operation 402. For example, over the time period that each breath sample is collected, the respective sensors of the BAIID capture multiple measurements (such as every 50 ms). These sensor measurements form a time series signal for each respective sensor type. For example, time-series sensor measurements for temperature, humidity, flow, pressure, proximity, and/or acoustics (e.g., sound) may be generated for each captured breath sample over the time for which the breath was being delivered. In some examples, the time-series sensor measurements include at least two, three, four, five, or all six of the above sensor measurement types.

Generating the baseline measurements may include fitting baseline curves to each of the time-series sensor measurements that are generated. The fitted baseline curves may account for the multiple captured baseline breath samples. Other characteristics of the captured baseline sensor measurements may be extracted and used for later comparison, such as maxima, minima, average values, and/or rates of change, among other features.

At operation 408, a breath is received for a subsequent breath test with the BAIID. For instance, the subsequent breath test may be an initial test or a rolling retest, as discussed above. During the receipt of the breath, the respective sensor measurements are captured. At operation 410, time-series sensor measurements are generated for the test breath captured at operation 408.

At operation 412, a determination is made as to whether the time-series sensor measurements for the test breath (generated in operation 410) deviate from the baseline time-series sensor measurements (generated in operation 406). The determination may be based on a comparison of the baseline curve(s) to time-series sensor measurements of the current breath to identify deviations. If the deviations exceed a deviation threshold, the test breath may be considered to deviate from the baseline, indicating a potential surrogate circumvention. The determination in operation 412 may also include comparison of the baseline characteristics for the baseline breaths to the corresponding characteristics of the test breath. For instance, maxima, minima, and/or rates of change of the respective signals may be compared to one another to identify differences. If the differences in the characteristics exceed a deviation threshold, the test breath may be considered to deviate from the baseline, indicating a potential surrogate circumvention.

If the test breath does not deviate from the baseline at operation 412, the method 400 flows back to operation 408 where a later breath test is performed and the subsequent operations repeat for the subsequent test. If the test breath does deviate from the baseline at operation 412, the method 400 flows to operation 414.

At operation 414, a surrogate check is performed based on the test breath deviating from the baseline. The surrogate check may include performing operations similar to operations 336-340 of method 300 described above. For instance, an image captured during the breath test may be compared to a stored photo of the assigned offender to determine if the user that delivered the test breath is the same person as the assigned offender. If the user is not the assigned offender, a surrogate circumvention may be identified and reported to the monitoring authority.

FIG. 5 depicts another example method 500 of triggering a surrogate check based on interlock data. By identifying the likely surrogate circumvention events using the methods 300 and 400 discussed above, a large amount of sensor and test data may be collected and used for the training of machine learning (ML) models that can then alternatively or additionally be used to identify potential surrogate circumvention events. For instance, the sensor data, timing data, and/or pass/fail/abort data of the respective tests may be used as training data for a supervised training process. Method 500 depicts an example process for performing such training and implementing the trained ML model.

At operation 502, the interlock data associated with the identified surrogate circumvention events is labeled as being associated with a surrogate circumvention event. The interlock data may include the sensor data (e.g., time-series measurements), the alcohol level recorded, whether the test was passed, failed, or aborted, among other potential test data. The time of the test(s) may also be included. The interlock data may be for multiple tests that led to the identification of surrogate circumvention, such as the first initial test, the second initial test, and/or the rolling retest, as discussed above with respect to method 300 in FIGS. 3A-3B.

At operation 504, interlock data associated with breath tests that were not surrogate circumvention attempts are labeled as normal tests or non-surrogate-circumvention data. The data may be substantially the same type of data as discussed above.

At operation 506, a supervised training of an ML model is performed with the training data labeled in operations 502 and 504. The ML model may be one of many different possible models, such as regression or classification models. These may include decision trees, support vector machines (SVMs), neural networks, etc.

The supervised training may include initializing the ML with starting or initialization parameters. The training data is then provided into the model to generate predictions. A loss or error between the prediction and the known label is then calculated through a loss function. The parameters are then adjusted until an acceptable loss or error is achieved. This process may occur for many iterations until the ML generates suitable predictions.

The trained ML model may then be implemented for live breath data. At operation 508, breath data is received for one or more live breath tests, such as initial tests and/or rolling retests. The breath data may include similar types of data on which the ML model was trained, such as sensor measurements and/or test results (e.g., pass, fail, abort). The breath data may also be for multiple breath tests, such as a combination of multiple initial tests and/or rolling retests (e.g., first initial test, second initial test, and rolling retest).

At operation 510, the live breath data is provided as input to the trained ML model. The ML model processes the received live breath data and generates an output. The output is received in operation 512. The output includes a prediction of whether the live breath data corresponds to a surrogate circumvention event. The prediction may be in the form of a confidence score or probability, such as a probability between 0-1, with a value of 1 indicating a high likelihood of a surrogate circumvention event.

At operation 514, a determination is made as to whether the received live breath data corresponds to a likely surrogate circumvention event. The determination is made based on the output of the ML model received in operation 512. For instance, the probability within the output may be compared to a threshold (e.g., 0.8). If the probability exceeds the threshold, there is likely a surrogate circumvention event. If the probability does not exceed the threshold, it is unlikely that there is a surrogate circumvention event.

If there is no surrogate circumvention event detected in operation 514, method 500 flows back to operation 508 where operations 508-516 can be repeated for subsequent live breath tests. If there is a likely surrogate circumvention event associated with the live breath data, method 500 flows to operation 516 where a surrogate check is triggered. The surrogate check may be performed as discussed above, such as by comparing captured images with one another and/or with a stored image of the assigned offender to determine if a surrogate delivered one or more breaths for the corresponding live breath tests.

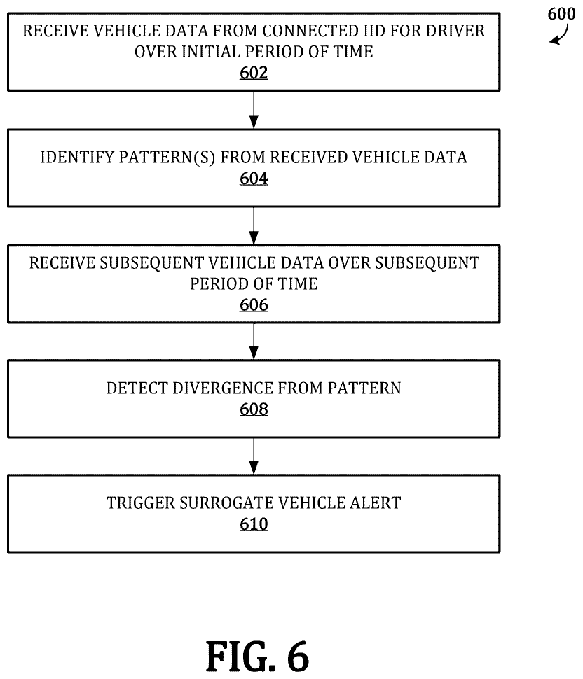

FIG. 6 depicts an example method 600 of triggering a surrogate vehicle alert. While surrogate circumvention described above relates to a surrogate human performing a breath test in place of the assigned offender, another type of circumvention is a surrogate vehicle circumvention. In this type of circumvention, the assigned offender starts using a vehicle other than the vehicle with the BAIID installed. Accordingly, the offender may continue to drive the other vehicle (e.g., surrogate vehicle) without having to first pass a breath test. The technology described in this application can detect a likely surrogate vehicle circumvention as well, and method 600 provides an example of such a process.

At operation 602, vehicle data is received from a connected BAIID for a driver (e.g., assigned offender) over an initial period of time. The vehicle data may include times at which the vehicle ignition and/or engine is started and stopped. The vehicle data may also include the speed of the vehicle and/or locations traveled over the initial time period. The vehicle data may be represented in drive cycles. Each drive cycle may correspond to a start of the vehicle and a stop of the vehicle (e.g., each time the vehicle is operated). The drive-cycle data may thus include start time, stop time, and/or duration of the drive cycle.

The initial time period may be on the order of weeks or months to allow for capturing of patterns of drive cycles. For instance, the initial time period may be between 1-3 weeks. In other examples, the time period may be between 2-6 weeks or 1-2 months.

At operation 604, a driving pattern is identified from the vehicle data received in operation 602. The driving pattern corresponds to repeated driving characteristics in the vehicle data, such as repetitions in drive cycles. These patterns may be identified through various pattern-extraction algorithms, such as regression algorithms, clustering algorithms (e.g., K-means, hierarchical), association rule learning, and/or principal component analysis (PCA), among others. The pattern may also include the number of drive cycles per week or per day (or other time period). The time of the drive cycles may also be identified (e.g., one drive cycle every morning and every evening).

At operation 606, subsequent vehicle data is received over a subsequent period of time after the initial period of time. The subsequent vehicle data may include the same type of data as the vehicle data received in operation 602, such as drive-cycle data.

At operation 608, a divergence of the subsequent vehicle data from the identified pattern is detected. The divergence may be a reduction in drive cycles from the pattern. For example, an average number of drive cycles in the subsequent vehicle data may be less than the average number of drive cycles in the identified pattern. Other divergences are also possible, such as a change in the time of drive cycles. The divergence from the pattern indicates that the assigned offender is likely driving another vehicle instead of the vehicle with the installed BAIID.

Based on identifying the divergence from the pattern in operation 608, a surrogate vehicle alert is triggered in operation 610. The surrogate vehicle alert may include a message generated to the monitoring authority. The message may include data regarding the pattern and the identified divergence. The monitoring authority may then contact the assigned offender to inquire about the divergence from the usual pattern.

While the techniques and procedures in methods depicted in FIGS. 3-6 are depicted and/or described in a certain order for purposes of illustration, it should be appreciated that certain procedures may be reordered and/or omitted within the scope of various embodiments. The operations of the method described therein may also be performed by one or more components of system 100, among other types of computing devices.

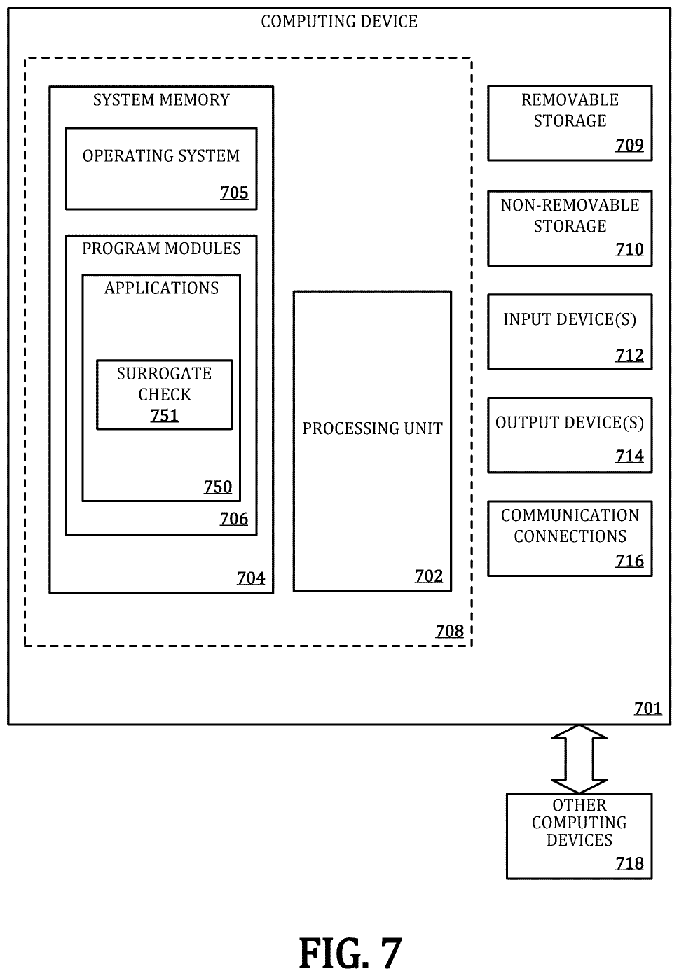

FIG. 7 is a block diagram illustrating physical components (e.g., hardware) of a computing device 701 with which examples of the present disclosure may be practiced. The computing device components described below may be suitable for one or more of the components of the system 100 described above. For instance, one or more of the remote servers may include one or more computing devices such as computing device 701.

In a basic configuration, the computing device 701 includes at least one processing unit 702 and a system memory 704. Depending on the configuration and type of computing device 701, the system memory 704 may comprise volatile storage (e.g., random access memory), non-volatile storage (e.g., read-only memory), flash memory, or any combination of such memories. The system memory 704 may include an operating system 705 and one or more program modules 706 suitable for running software applications 750 (e.g., surrogate check application 751) and other applications.

The operating system 705 may be suitable for controlling the operation of the computing device 701. Furthermore, aspects of the disclosure may be practiced in conjunction with a graphics library, other operating systems, or any other application program and are not limited to any particular application or system. This basic configuration is illustrated in FIG. 7 by those components within a dashed line 708. The computing device 701 may have additional features or functionality. For example, the computing device 701 may also include additional data storage devices (removable and/or non-removable) such as, for example, magnetic disks, optical disks, or tape. Such additional storage is illustrated in FIG. 7 by a removable storage device 709 and a non-removable storage device 710.

As stated above, a number of program modules and data files may be stored in the system memory 704. While executing on the processing unit 702, the program modules 706 may perform processes including one or more of the operations of the methods and processes discussed herein, such as the methods of FIGS. 3-6. Other program modules that may be used in accordance with examples of the present disclosure may include additional applications as well.

Furthermore, examples of the disclosure may be practiced in an electrical circuit comprising discrete electronic elements, packaged or integrated electronic chips containing logic gates, a circuit utilizing a microprocessor, or on a single chip containing electronic elements or microprocessors. For example, examples of the disclosure may be practiced via a system-on-a-chip (SOC) where each or many of the components illustrated in FIG. 7 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to detecting an unstable resource may be operated via application-specific logic integrated with other components of the computing device 701 on the single integrated circuit (chip). Examples of the present disclosure may also be practiced using other technologies capable of performing logical operations such as, for example, AND, OR, and NOT, including mechanical, optical, fluidic, and quantum technologies.

The computing device 701 may also have one or more input device(s) 712 such as a keyboard, a mouse, a pen, a sound input device, a touch input device, a camera, etc. The output device(s) 714 such as a display, speakers, a printer, etc. may also be included. The aforementioned devices are examples and others may be used. The computing device 701 may include one or more communication connections 716 allowing communications with other computing devices 718. Examples of suitable communication connections 716 include RF transmitter, receiver, and/or transceiver circuitry; universal serial bus (USB), parallel, and/or serial ports.

The term computer readable media as used herein includes volatile and nonvolatile, removable and non-removable media implemented in any method or technology for storage of information, such as computer readable instructions, data structures, or program modules. The system memory 704, the removable storage device 709, and the non-removable storage device 710 are all computer readable media examples (e.g., memory storage.) Computer readable media include random access memory (RAM), read-only memory (ROM), electrically erasable programmable ROM (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 701. Any such computer readable media may be part of the computing device 701. Computer readable media does not include a carrier wave or other propagated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, communication media may include wired media such as a wired network or direct-wired connection, and wireless media such as acoustic, RF, infrared, and other wireless media.

It is to be understood that the methods, modules, and components depicted herein are merely examples. Alternatively, or in addition, the functionality described herein can be performed, at least in part, by one or more hardware logic components. For example, illustrative types of hardware logic components that can be used include Field-Programmable Gate Arrays (FPGAs), Application-Specific Integrated Circuits (ASICs), Application-Specific Standard Products (ASSPs), System-on-a-Chip systems (SOCs), Complex Programmable Logic Devices (CPLDs), etc. In an abstract, but still definite sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or inter-medial components. Likewise, any two components so associated can also be viewed as being “operably connected,” or “coupled,” to each other to achieve the desired functionality. Merely because a component, which may be an apparatus, a structure, a system, or any other implementation of a functionality, is described herein as being coupled to another component does not mean that the components are necessarily separate components. As an example, a component A described as being coupled to another component B may be a sub-component of the component B, the component B may be a sub-component of the component A, or components A and B may be a combined sub-component of another component C.

The functionality associated with some examples described in this disclosure can also include instructions stored in a non-transitory media. The term “non-transitory media” as used herein refers to any media storing data and/or instructions that cause a machine to operate in a specific manner. Illustrative non-transitory media include non-volatile media and/or volatile media. Non-volatile media include, for example, a hard disk, a solid-state drive, a magnetic disk or tape, an optical disk or tape, a flash memory, an EPROM, NVRAM, PRAM, or other such media, or networked versions of such media. Volatile media include, for example, dynamic memory such as DRAM, SRAM, a cache, or other such media. Non-transitory media is distinct from, but can be used in conjunction with, transmission media. Transmission media is used for transferring data and/or instruction to or from a machine. Examples of transmission media include coaxial cables, fiber-optic cables, copper wires, and wireless media, such as radio waves.

Furthermore, those skilled in the art will recognize that boundaries between the functionality of the above-described operations are merely illustrative. The functionality of multiple operations may be combined into a single operation, and/or the functionality of a single operation may be distributed in additional operations. Moreover, alternative embodiments may include multiple instances of a particular operation, and the order of operations may be altered in various other embodiments.

Although the disclosure provides specific examples, various modifications and changes can be made without departing from the scope of the disclosure as set forth in the claims below. Accordingly, the specification and figures are to be regarded in an illustrative rather than a restrictive sense, and all such modifications are intended to be included within the scope of the present disclosure. Any benefits, advantages, or solutions to problems that are described herein with regard to a specific example are not intended to be construed as a critical, required, or essential feature or element of any or all the claims.

Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to containing only one such element, even when the same claim includes the introductory phrases “one or more” or “at least one” and indefinite articles such as “a” or “an.” The same holds true for the use of definite articles.

Unless stated otherwise, terms such as “first” and “second” are used to arbitrarily distinguish between the elements such terms describe. Thus, these terms are not necessarily intended to indicate temporal or other prioritization of such elements.

What is claimed is:

1. A method for data-driven surrogate circumvention detection, the method comprising:
receiving, by a breath alcohol ignition interlock device (BAIID), a first breath for a first initial test;
capturing a first image of a user that delivered the first breath;
measuring, by the BAIID, a first alcohol content in the first breath;
determining, by the BAIID, that the first alcohol content fails the first initial breath test;
receiving, by the BAIID, a second breath for a second initial breath test;
capturing a second image of a user that delivered the second breath;
measuring, by the BAIID, a second alcohol content of the second breath;
determining, by the BAIID, that the second alcohol content passes the second initial breath test;
initiating, by the BAIID, a rolling retest;
determining, by the BAIID, that the rolling retest has either been skipped or failed; and
based on the failing of the first initial test, the passing of the second initial test, and the skipping or failing of the rolling retest, triggering a surrogate check.

2. The method of claim 1, further comprising performing the surrogate check by performing an image comparison with the captured first image and the captured second image.

3. The method of claim 2, wherein the image comparison compares the first image to the second image to determine if the user that delivered the first breath is the same person as the user that delivered the second breath.

4. The method of claim 2, wherein the image comparison compares at least one of the first image or the second image to a stored photo of a person to which the BAIID was assigned.

5. The method of claim 2, wherein the image comparison is performed via a facial recognition algorithm.

6. The method of claim 2, wherein the surrogate check indicates that a surrogate circumvention event occurred, and the method further comprises generating a message to a monitoring authority indicating the occurrence of the surrogate circumvention event.

7. The method of claim 1, wherein the rolling retest is skipped.

8. The method of claim 1, wherein the rolling retest is failed, and the method further comprises:
receiving, by the BAIID, a third breath for the rolling retest;
capturing a third image of a user delivering the third breath; and
measuring, by the BAIID, a third alcohol content of the third breath.

9. The method of claim 8, further comprising performing the surrogate check by performing an image comparison with at least two of the first image, the second image, and the third image.

10. The method of claim 1, wherein triggering of the surrogate check is further based on the failing of the first initial test, the passing of the second initial test, and the skipping or failing of the rolling retest all occurring within a threshold timespan.

11. The method of claim 10, wherein the threshold timespan is between 0-3 hours.

12. A method for data-driven surrogate circumvention detection, the method comprising:
collecting, by a breath alcohol ignition interlock device (BAIID), baseline breath samples;
generating baseline time-series sensor measurements from the collected baseline breath samples, wherein the baseline time-series sensor measurements are based on a time-series of measurements made by sensors of the BAIID during collection of the baseline breath samples;
receiving, by the BAIID, a breath sample for a subsequent breath test;
generating, by the BAIID, test time-series sensor measurements for the subsequent test breath;
comparing the test time-series sensor measurements to the baselines time-series sensor measurements; and
based on the comparison, triggering a surrogate check.

13. The method of claim 12, wherein the baseline time-series sensor measurements and the test time-series sensor measurements include measurements from at least one of a temperature sensor of the BAIID, a humidity sensor of the BAIID, a flow sensor of the BAIID, a pressure sensor of the BAIID, a proximity sensor of the BAIID, or an acoustic sensor of the BAIID.

14. The method of claim 13, wherein the baseline time-series sensor measurements and the test time-series sensor measurements include measurements from at least two of the temperature sensor of the BAIID, the humidity sensor of the BAIID, the flow sensor of the BAIID, the pressure sensor of the BAIID, the proximity sensor of the BAIID, or the acoustic sensor of the BAIID.

15. The method of claim 14, wherein the baseline time-series sensor measurements and the test time-series sensor measurements include measurements from at least two of the temperature sensor of the BAIID, the humidity sensor of the BAIID, or the flow sensor of the BAIID.

16. The method of claim 15, wherein the baseline time-series sensor measurements and the test time-series sensor measurements include measurements from the temperature sensor of the BAIID, the humidity sensor of the BAIID, the flow sensor of the BAIID, the pressure sensor of the BAIID, the proximity sensor of the BAIID, and the acoustic sensor of the BAIID.

17. The method of claim 12, further comprising:
capturing an image of a user delivering the breath sample for the subsequent breath test; and
performing the surrogate check by performing an image comparison with the captured image.

18. A method for data-driven surrogate vehicle circumvention detection, the method comprising:
receiving, from a breath alcohol ignition interlock device (BAIID), vehicle data for an initial period of time;
identifying a pattern in the vehicle data received during the initial period of time;
receiving, from the BAIID, subsequent vehicle data over a subsequent period of time;
detecting a divergence of the subsequent vehicle data from the identified pattern; and
based on detecting the divergence, triggering a surrogate vehicle alert.

19. The method of claim 18, wherein identifying the pattern includes determining a frequency of drive cycles.

20. The method of claim 19, wherein detecting the divergence includes detecting a reduction in the number of drive cycles in the subsequent period of time as compared to the initial period of time.
